(19) United States
(12) Patent Application Publication
(10) Pub. No.: US 2002/0133251 A1
(43) Pub. Date: Sep. 19, 2002

(54) Dynamic techniques for custom-fit knee replacments

(75) Inventors: Menachem Levanoni, Yorktown Heights, NY (US); Jerome M. Kurtzberg, Yorktown Heights, NY (US)

Correspondence Address: Stephen C. Kaufman, Intellectual Property Law Dept., IBM Corporation, P.O. Box 218, Yorktown Heights, NY, 10598, US

(73) Assignee: International Business Machines Corporation,, Armonk, NY

(21) Appl. No.: 09/809,577

(22) Filed: Mar. 15, 2001

(51) Int. Cl. G06F 19/00; A61B 1/00; A61B 1/04

(52) U.S. Cl. 700/97; 600/117

(57) ABSTRACT

The invention discloses methodology for utilizing continual sensor-based data to design and adjust orthodics to fit an individual, in a given dynamic environment, preferably in an optimal manner. The method includes procedures for producing a virtual orthodic so that an actual construction of a physical orthodic can be directly obtained from the virtual orthodic.

### BACKGROUND OF THE INVENTION

1. Field of the Invention

This invention relates to methodology for utilizing continual sensor-based data to design and adjust orthodics to fit an individual, in a given dynamic environment, preferably in an optimal manner.

2. Introduction to the Invention

Static fitting techniques to design and construct orthodics for specific people are known. A plaster cast is taken and the orthodic is produced based on that plastic impression. We note, however, that no attention is given to the dynamic workings of the knee in the changing real environment. Specifically, the stresses and accelerations experienced by the knee during normal operation are not taken into account, nor is an optimum balance, between support and comfort, taken into account.

### SUMMARY OF THE INVENTION

We have now discovered novel methodology for exploiting the advantages inherent generally in sensing the dynamic workings (stresses) on specific knees/hips in actual motion, and using the sensor-based data to optimize the design and construction of the desired orthodics.

Our work proceeds in the following way.

We have recognized that a typical and important paradigm for presently effecting orthodics construction, is a largely static and subjective, human paradigm, and therefore exposed to all the vagaries and deficiencies otherwise attendant on static and human procedures. Instead, the novel paradigm we have in mind works in the following way:

First, a patient wears a set of pressure and acceleration sensors mounted, say, inside a knee-encasing device. These sensors record their associated stresses and accelerations produced in normal individual motion in its dynamic environment for a prescribed period of time sufficient to capture all possible stress and acceleration patterns.

The dynamically acquired data are fed into a computer which creates a map of the forces and accelerations experienced by the examined knee. This information is used to design an optimal orthodic which maximizes support and minimizes discomfort, and results in a computer production of a virtual orthodics that offers optimal performance to the examined knee in its normal operation.

A physical orthodic is then produced from a model provided by the virtual orthodic. This physical orthodic provides maximum support and maximal comfort to its wearer, following the optimal design of the orthodic.

We now disclose a novel computer method which can preserve the advantages inherent in the static approach, while minimizing the incompleteness and attendant static nature and subjectivities that otherwise inure in a technique heretofore used.

To this end, in a first aspect of the present invention, we disclose a novel computer method comprising the steps of:

i) providing pressure and acceleration sensors;

ii) mounting said sensors in a knee-enclosing device;

iii) transmitting data produced by said sensors during actual operation of said knee-enclosing device worn by a specific individual;

iv) receiving said sensor signals for subsequent analysis by a computer;

v) creating a stress-and-acceleration map based on said sensor-based data;

vi) creating a virtual orthodic (model) for support and comfort based on step v) stress-and-acceleration map; and

vii) constructing a physical orthodic based on a design provided by the virtual orthodic.

### BRIEF DESCRIPTION OF THE DRAWING

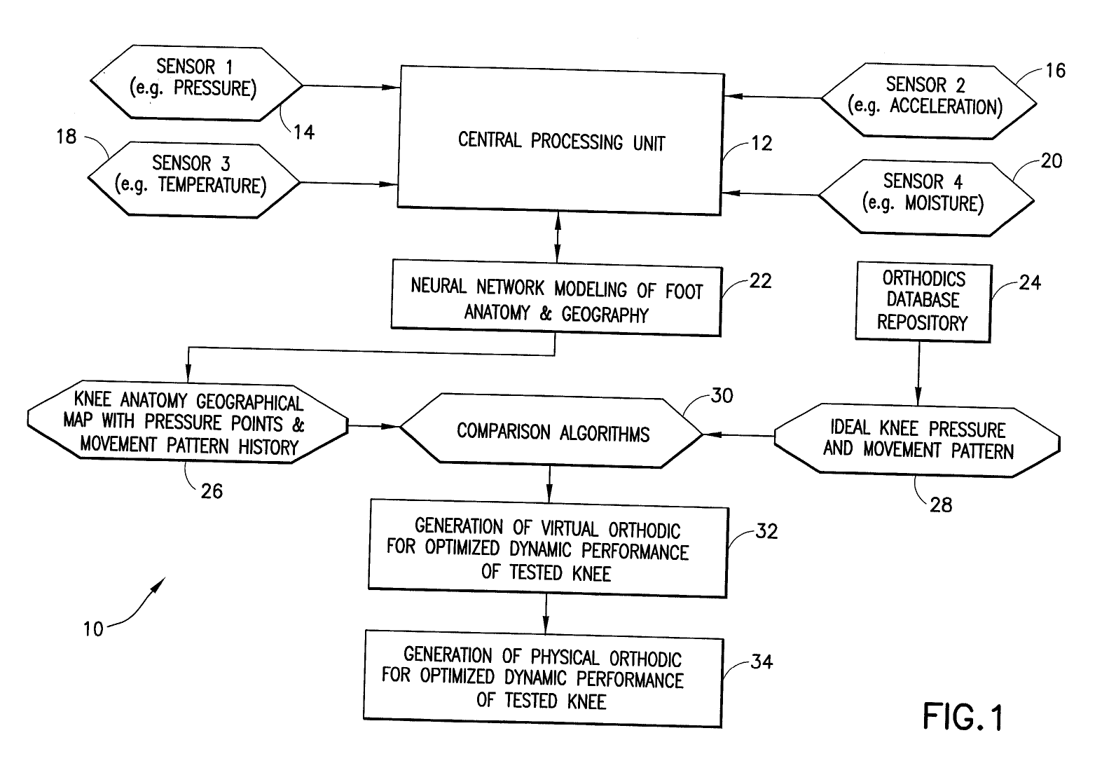

The invention is illustrated in the accompanying drawing, in which FIG. 1 provides an illustrative flowchart comprehending overall realization of the method of the present invention.

### DETAILED DESCRIPTION OF THE PRESENT INVENTION

The detailed description of the present invention is now disclosed, and in this regard, attention may be turned to the illustrative FIG. 1 (in numerals 10-34) which provides a flow-chart comprehending one way for realizing the method of the present invention.

In a typical case, the patient's knee is fitted with a temporary device containing a number of sensors, located at prescribed locations on the tested knee. These sensors, which preferably include pressure, acceleration, temperature, and humidity, are connected to a recording device.

The patient is asked to wear the device for several days and follow his/her normal routine.

During the test period, the sensor data is recorded (including time stamps) in the recording device. The patient returns the device and the recording device at the end of the test period. The information stored in the recording device is then downloaded to a computer which stores all data in a database.

The data are then analyzed by a program (preferably a neural network modeling program) which creates maps of the tested knee at different times. These maps also contain the sensors' reading at these times. Thus, the system now has information on the dynamic behavior of the tested knee, including parametric information.

Based on these maps and maps of an ideal knee under similar conditions, an optimization program designs an optimized virtual orthodic for the patient. This design is then fed to a machine which generates an optimized physical orthodic.

What is claimed:

1. A computer method comprising the steps of:
i) providing pressure and acceleration sensors;
ii) mounting said sensors in a knee-enclosing device;
iii) transmitting the data produced by said sensors during actual operation of said knee-enclosing device worn by a specific individual;
iv) receiving said sensor signals for subsequent analysis by a computer;
v) creating a stress-and-acceleration map based on said sensor-based data;
vi) creating a virtual orthodic (model) for support and comfort based on step v) stress-and-acceleration map; and
vii) constructing a physical orthodic based on a design provided by the virtual orthodic.

2. A method according to claim 1, comprising a step of using temperature, moisture, and skin conductivity sensors which are correlated with a worn orthodic.

3. A method according to claim 1, comprising a step of using interpolation techniques to completely map stresses and accelerations experienced by a knee over a period of time.

4. A method according to claim 3, comprising a step of updating the virtual orthodic model using the interpolating map.

5. A method according to claim 4, comprising a step of using the interpolated map to directly design the virtual orthodic in an optimal manner.

6. A method according to claim 1, comprising a step of using non-linear techniques to model an optimal orthodic.

7. A method according to claim 6, comprising a step of employing neural networks as the modeling technique.

8. A method according to claim 7, comprising a step of employing regression as the modeling technique.

9. A method according to claim 7, comprising a step of employing expert systems or fuzzy logic as the modeling technique.

10. A method according to claim 1, comprising the step of optimizing the design of the virtual orthodic subject to internal or external constraints.

11. A program storage device readable by machine, tangibly embodying a program of instructions executable by the machine to perform method steps for providing an interactive supply chain management database, the method comprising the steps of:
i) providing pressure and acceleration sensors;
ii) mounting said sensors in a knee-enclosing device;
iii) transmitting the data produced by said sensors during actual operation of said knee-enclosing device worn by a specific individual;
iv) receiving said sensor signals for subsequent analysis by a computer;
v) creating a stress-and-acceleration map based on said sensor-based data;
vi) creating a virtual orthodic (model) for support and comfort based on step v) stress-and-acceleration map; and
vii) constructing a physical orthodic based on a design provided by the virtual orthodic.
